(19) United States
(12) Patent Application Publication
(10) Pub. No.: US 2013/0345615 A1
(43) Pub. Date: Dec. 26, 2013

(54) Intracorneal Drug Delivery Device and Associated Methods

(71) Applicant: John W. Higuchi, Salt Lake City, UT (US)

(72) Inventor: John W. Higuchi, Salt Lake City, UT (US)

(21) Appl. No.: 13/529,873

(22) Filed: Jun. 21, 2012

(51) Int. Cl. A61F 9/00; A61N 1/30; A61M 35/00

(52) U.S. Cl. 604/20; 604/301; 604/290

(57) ABSTRACT

Methods and devices for delivering an active agent into the eye of a subject are provided. In one aspect, an ocular device for delivering an active agent through a cornea of an eye of a subject can include a housing configured to couple to the eye of the subject and a corneal seal coupled to the housing and positioned in the housing to encircle the cornea during use to form a corneal region. The device can also include an active agent reservoir coupled to the housing and positioned to release active agent into the corneal region, and a pressure regulator coupled to the housing and operable to introduce negative pressure between the housing and the eye. The corneal seal is operable to fluidically isolate the corneal region from the sclera in response to the negative pressure, thus minimizing contact of the active agent with the sclera.

### FIELD OF THE INVENTION

The present invention relates to systems, methods, and devices for the delivery of an active agent through a region of a subject's ocular tissue. Accordingly, the present invention involves the fields of chemistry, pharmaceutical sciences, and medicine, particularly ophthalmology.

### BACKGROUND OF THE INVENTION

Treatments of various eye conditions often require intravitreal and periocular injections or systemic drug administration. Systemic administration is usually not preferred because of the resulting systemic toxicity as discussed above. While intravitreal and periocular injections are preferable to systemic administration, the half-life of most injected compounds in the vitreous is relatively short, usually on the scale of just a few hours. Therefore, intravitreal injections require frequent administration. The repeated injections can cause pain, discomfort, intraocular pressure increases, intraocular bleeding, increased chances for infection, and the possibility of retinal detachment. The major complication of periocular injections is accidental perforation of the globe, which causes pain, retinal detachment, ocular hypertension, and intraocular hemorrhage. Other possible complications of periocular injections include pain, central retinal artery/vein occlusion, and intraocular pressure increases. Therefore, these methods of ocular drug delivery into the eye have significant limitations and major drawbacks. In addition, injections are very poorly accepted by patients. These methods also involve high healthcare cost due to the involvement of skilled and experienced physicians to perform the injections.

Ocular iontophoresis is a noninvasive technique used to deliver compounds of interest into the interior of a patient's eye. In practice, two iontophoretic electrodes are used in order to complete an electrical circuit. In traditional, transscleral iontophoresis, at least one of the electrodes is considered to be an active iontophoretic electrode, while the other may be considered as a return, inactive, or indifferent electrode. The active electrode is typically placed on an eye surface, and the return electrode is typically placed remote from the eye, for example on the earlobe. The compound of interest is transported at the active electrode across the tissue when a current is applied to the electrodes. Compound transport may occur as a result of a direct electrical field effect (e.g., electrophoresis), an indirect electrical field effect (e.g., electroosmosis), electrically induced pore or transport pathway formation (electroporation), or a combination of any of the foregoing. Examples of currently known iontophoretic devices and methods for ocular drug delivery may be found in U.S. Pat. Nos. 6,319,240; 6,539,251; 6,579,276; 6,697,668, and PCT Publication Nos. WO 03/030989 and WO 03/043689, each of which is incorporated herein by reference.

The problem of patient compliance may be compounded by the need to receive daily treatment in a medical facility with high healthcare costs and limited resources and practitioners for treating retinal diseases. Existing ocular iontophoresis systems are not patent-friendly, require multiple parts and assembly to practice, and include either clumsy or complicated procedures. As such, they require the involvement of experienced healthcare professionals to perform the treatments. Paraprofessional and/or in-home self administration use of such devices are precluded by the technical complexity of many existing iontophoretic devices, as well as the costs of expensive dose-controlling equipment. Individuals have a greater tendency to deviate from a medication regimen when required to leave home for medical treatment, particularly when such treatment is frequent.

### SUMMARY OF THE INVENTION

The present disclosure provides methods and devices for the ocular delivery of an active agent. In one aspect, for example, an ocular device for delivering an active agent through a cornea of an eye of a subject is provided. Such a device can include a housing configured to couple to the eye of the subject and a corneal seal coupled to the housing and positioned in the housing to encircle the cornea during use to form a corneal region. The device can also include an active agent reservoir coupled to the housing and positioned to release active agent into the corneal region, and in some aspects only or substantially only to the corneal region, and a pressure regulator coupled to the housing and operable to introduce negative pressure between the housing and the eye. The corneal seal is operable to fluidically isolate the corneal region from the sclera in response to the negative pressure, thus minimizing contact of the active agent with the sclera. In one specific aspect, the active agent reservoir can be fluidically coupled to the pressure regulator such that the active agent is released into the corneal region as a result of activation of the pressure regulator. In another specific aspect, the active agent can be structurally configured to release the active agent into a secondary active agent reservoir located in the corneal region. In yet another aspect, the housing extends outward from the corneal seal to form a scleral region positioned over the eye's sclera during use.

Various pressure regulator configurations are contemplated, and any such configuration that allows negative pressure to be introduced between the eye and the housing is considered to be within the present scope. In one aspect, for example, the pressure regulator can be a vacuum bulb. In another aspect, the pressure regulator can be structurally configured to release active agent from the active agent reservoir as the vacuum bulb is depressed and the corneal seal can be configured to seal to the eye as the vacuum bulb is released. In yet another aspect, the pressure regulator can be operable to introduce positive pressure between the housing and the eye to facilitate release of the housing from the eye. In a further aspect, the pressure regulator can be positioned in the housing sufficiently below a midline of the housing such that the subject can substantially close the eye during delivery of the active agent.

It is contemplated that the present device can be utilized for passive and/or active delivery of an active agent into the eye. In one aspect, for example, the device can include an anode and a cathode both positioned to be facing the eye and at least one of the anode and cathode being in fluid communication with the active agent reservoir during use. In one specific aspect, the active agent reservoir can include an active agent. In another specific aspect, the active agent can be resiniferatoxin.

The present disclosure additionally provides a method of anesthetizing a cornea of an eye of a subject while leaving the sclera substantially unanesthetized. Such a method can include applying a corneal seal to the eye to encircle the cornea to form a corneal region, and applying a negative pressure between the housing and the eye to fluidically isolate the corneal region from the sclera. The method can also include delivering an anesthetic agent to the corneal region to anesthetize the cornea, where the active agent is substantially precluded from the sclera by the corneal seal. In one specific aspect, the anesthetic agent is a vanilloid receptor agonist. In another aspect, the vanilloid receptor agonist is resiniferatoxin.

The present disclosure also provides a method of delivering an active agent through a cornea of an eye of a subject while minimizing delivery of the active agent through the eye's sclera. Such a method can include applying a corneal seal to the eye to encircle the cornea to form a corneal region, and applying a negative pressure between the housing and the eye to fluidically isolate the corneal region from the sclera. The method can also include delivering an active agent to the corneal region, where the active agent is substantially precluded from the sclera by the corneal seal. In another aspect, applying the negative pressure further includes applying a positive pressure to deliver the active agent into the corneal region followed by applying the negative pressure between the housing and the eye to fluidically isolate the corneal region from the sclera. In one specific aspect, the negative pressure can be applied between the housing and the eye in the sclera. In another specific aspect, the negative pressure is applied between the housing and the eye in the corneal region. In yet another specific aspect, the method can further include applying a positive pressure between the housing and the eye to facilitate release of the housing from the eye. In a further specific aspect, delivering the active agent includes delivering the active agent passively to the corneal region. In yet a further specific aspect, delivering the active agent includes delivering the active agent iontophoretically to the corneal region.

### BRIEF DESCRIPTION OF THE DRAWINGS

FIG. 1 is a cross section view of an active agent delivery device in accordance with an aspect of the present invention.

FIG. 2 is a cross section view of an active agent delivery device in accordance with another aspect of the present invention.

FIG. 3 is a cross section view of an active agent delivery device in accordance with yet another aspect of the present invention.

FIG. 4 is a cross section view of an active agent delivery device in accordance with a further aspect of the present invention.

FIG. 5 is a cross section view of an active agent delivery device in accordance with a further aspect of the present invention.

FIG. 6 is a cross section view of an active agent delivery device in accordance with a further aspect of the present invention.

### DETAILED DESCRIPTION OF THE INVENTION

Before the present devices and methods for ocular drug delivery are disclosed and described, it is to be understood that this invention is not limited to the particular process steps and materials disclosed herein, but is extended to equivalents thereof, as would be recognized by those ordinarily skilled in the relevant arts. It should also be understood that terminology employed herein is used for the purpose of describing particular embodiments only and is not intended to be limiting.

It must be noted that, as used in this specification and the appended claims, the singular forms “a,” “an,” and, “the” include plural referents unless the context clearly dictates otherwise. Thus, for example, reference to “a polymer” includes reference to one or more of such polymers, and “an excipient” includes reference to one or more of such excipients.

### Definitions

In describing and claiming the present invention, the following terminology will be used in accordance with the definitions set forth below.

As used herein, “formulation” and “composition” may be used interchangeably herein, and refer to a combination of two or more elements, or substances. In some embodiments a composition may include an active agent, an excipient, or a carrier to enhance delivery or depot formation.

As used herein, “active agent” may be used to refer to an agent or substance that has measurable specified or selected physiologic activity when administered to a subject in a significant or effective amount. It is to be understood that the term “drug” is expressly encompassed by the present definition as many drugs and prodrugs are known to have specific physiologic activities. These terms of art are well-known in the pharmaceutical, and medicinal arts. Examples of drugs useful in the present invention include without limitation, steroids, antibacterials, antivirals, antifungals, antiprotozoals, antimetabolites, immunosuppressive agents, VEGF inhibitors, ICAM inhibitors, antibodies, protein kinase C inhibitors, chemotherapeutic agents, neuroprotective agents, nucleic acid derivatives, aptamers, proteins, enzymes, peptides, polypeptides, vanilloid receptor agonists, and the like.

As used herein “prodrug” refers to a molecule that will convert into a drug (its commonly known pharmacological active form). Prodrugs themselves can also be pharmacologically active, and therefore are also expressly included within the definition of an “active agent” as recited above. For example, dexamethasone phosphate can be classified as a prodrug of dexamethasone, and triamcinolone acetonide phosphate can be classified as a prodrug of triamcinolone acetonide.

As used herein, “effective amount,” and “sufficient amount” may be used interchangeably and refer to an amount of an ingredient which, when included in a composition, is sufficient to achieve an intended compositional or physiological effect. Thus, a “therapeutically effective amount” refers to a non-toxic, but sufficient amount of an active agent, to achieve therapeutic results in treating a condition for which the active agent is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, “sclera” refers to the sclera tissue in the eye or the conjunctiva between the limbus and the formix on the surface of the eye, which is the white part of the eye. In some aspects “sclera” can be used in referring to other eye tissues.

As used herein, “eye” refers to the globe of the eye. As such, delivery of an active agent into the eye refers to delivery of the active agent into the globe of the eye itself.

As used herein, “subject” refers to a mammal that may benefit from the administration of a composition or method as recited herein. Examples of subjects include humans, and may also include other animals such as horses, pigs, cattle, dogs, cats, rabbits, and aquatic mammals.

As used herein, “administration,” and “administering” refer to the manner in which an active agent, or composition containing such, is presented to a subject. As discussed herein, the present invention is primarily concerned with noninvasive delivery such as passive delivery or iontophoretic delivery, especially with ocular delivery.

As used herein, “noninvasive” refers to a form of administration that does not rupture or puncture a biological membrane or structure with a mechanical means across which a drug or compound of interest is being delivered. A number of noninvasive delivery mechanisms are well recognized in the transdermal arts such as patches, and topical formulations. Many of such formulations may employ a chemical penetration enhancer in order to facilitate non-invasive delivery of the active agent. Additionally, other systems or devices that utilize a non-chemical mechanism for enhancing drug penetration, such as iontophoretic devices are also known. “Minimally invasive” refers to a form of administration that punctures a biological membrane or structure but does not cause excessive discomfort to the subjects and severe adverse effects. Examples of “minimally invasive” drug delivery are microneedle, laser, or heat punctuation in transdermal delivery and periocular injections in ocular delivery.

As used herein, the term “outward” refers to a direction extending away from the center of the cornea. Thus extending “outward” from the corneal seal is intended to describe a region extending from the side of the corneal seal opposite to the center of the cornea.

As used herein, the term “body surface” refers to an outer tissue surface of the subject such as tissue surfaces encountered in ocular and transdermal delivery, or mucosal tissues lining a body cavity such as the mouth for buccal delivery or vaginal tract for vaginal delivery. The term “skin” refers to an outer tissue surface of the subject. It is therefore intended that skin also refer to mucosal and epithelial tissues, as well as the outer surfaces of the eye.

As used herein, the terms “anode” and “cathode” refer to the electrical polarity of an electrode. The terms “anode” and “cathode” are well known in the art. It should be noted, however, that in some aspects these descriptive terms may be transitory. When using alternating current, for example, two electrodes will alternate between anode and cathode as the current alternates in electrical polarity.

As used herein, the term “reservoir” refers to a body, a lumen, or a mass that may contain an active agent, a depot forming agent, secondary compound, or other pharmaceutically useful compound or composition. As such, a reservoir may include any structure that may contain a liquid, a gelatin, a semi-solid, a solid or any other form of active agent or secondary compound known to one of ordinary skill in the art. In some cases, an electrode may be considered to be a reservoir.

As used herein, the term “contact lens” refers to a lens sized to fit approximately over the cornea of the eye.

As used herein, the term “scleral lens” refers to a lens sized to cover and extend beyond the cornea across at least a portion of the sclera of the eye.

As used herein, the term “active electrode” refers to an electrode utilized to iontophoretically deliver an active agent.

As used herein, the term “passive electrode” refers to an electrode that is used to complete an electrical circuit without delivering a compound or substance to a subject.

As used herein, the term “return electrode” refers to an electrode utilized to complete an electrical circuit for active electrode. In one aspect, a return electrode may be an active electrode used to deliver a secondary compound, such as an active agent, a depot forming agent, etc. In another aspect, a return electrode may be a passive electrode.

As used herein, the term “self-contained” refers to a device that contains therein, or substantially therein, all the components required for use. For example, a self-contained iontophoretic device may contain active agents, reservoirs, electrodes, power supplies, etc., within a single housing.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc.

This same principle applies to ranges reciting only one numerical value. Furthermore, such an interpretation should apply regardless of the breadth of the range or the characteristics being described.

### THE INVENTION

The present disclosure relates to devices for the delivery of an active agent through the cornea of the eye of a subject, including various methods associated with such devices. Various conditions can be treated by delivering an active agent to or through the cornea of the eye without substantial contact with the sclera. Additionally, in some cases it can be desirable to administer a pain reliever or anesthetic to the cornea but not to the sclera. The inventors have discovered devices and methods that facilitate such discrete delivery of an active agent into ocular tissue. For example, a corneal seal can be applied to the surface of the eye in order to isolate the cornea from the sclera during delivery of an active agent. In some aspects, the functionality of the corneal seal can be facilitated and/or enhanced through the use of a vacuum applied between the device housing and the surface of the eye. This vacuum force can function to improve contact between the seal and the eye, thus isolating the sclera from contact with the active agent, and precluding contact between the active agent and lachrymal fluid, among other things. By applying a vacuum force between the eye and the device, the device housing is brought into a more intimate contact with the surface of the eye. Such an improved contact can have a variety of beneficial effects pertaining to ocular drug delivery, and such benefits can vary depending on the design of the device and the location of the vacuum within the device and relative to the surface of the eye. For example, a vacuum between the device and the eye can cause a tight adherence there between. Such a tight adherence or “gripping” of the eye surface by the device maintains the active agent reservoir at a distinct location with respect to the eye. In some cases, eye tissue associated with a reservoir can become “primed” and thus more readily pass active agent there through. Accordingly, maintaining the reservoir at a distinct location can enhance active agent delivery.

As another example, when a device housing is placed upon the eye, air pockets can be present along the eye/device interface. This air can, in some cases, increase the required travel distance for an active agent to move to and contact the eye surface during delivery. In addition to increasing travel or diffusion distance, air situated between the eye and the device can greatly slow or even halt the movement of the active agent through the surrounding fluid. A vacuum force pulls the housing toward the eye, thus displacing a portion of the fluid and/or the trapped air. In those cases where the active agent is delivered from a portion of the housing subject to the vacuum, air pockets are eliminated or substantially reduced by the vacuum, and thus increased mobility of the active agent is achieved. In those cases where the active agent is delivered from a portion of the housing that is not subject to the vacuum (i.e. separated by a barrier structure), air pockets can be drawn to the vacuumed portion as the barrier seals and can be pressed from the interface as the housing is pulled toward the eye.

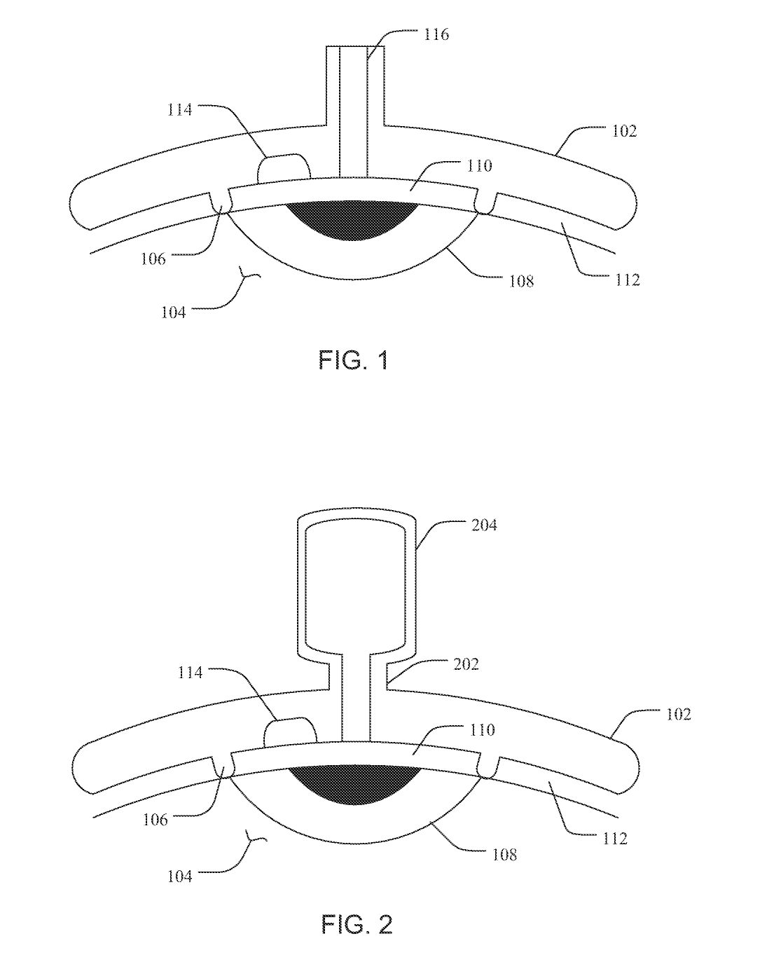

Accordingly, in one aspect of the present disclosure, an ocular device for delivering an active agent through a cornea of an eye of a subject is provided. As is shown in FIG. 1, such a device can include a housing 102 configured to couple to the eye of the subject, and a corneal seal 106 coupled to the housing 102 and positioned in the housing 102 to encircle the cornea 108 of a subject's eye, during use to form a corneal region 110. In some aspects, the housing 102 optionally extends outward from the corneal seal 106 to form a scleral region 112, where the scleral region 112 is positioned over the eye's sclera 104 or conjunctiva during use during use. The corneal region 110 is thus an area that is sealed from the scleral region 112 (or in some aspects where the housing does not extend over the sclera, the corneal region is sealed from the sclera). An active agent reservoir 114 can be coupled to the housing 102 and positioned to release active agent into the corneal region 110. The active agent reservoir can be located over a discrete portion of the cornea as shown, or it can be located across all or substantially all of the cornea. In other aspects, the active agent reservoir can include discrete channels, concentric channels, and the like. In some aspects, the corneal region can be the active agent reservoir.

A pressure regulator 116 can be coupled to the housing 102, and can thus be operable to introduce negative pressure (i.e. a vacuum) between the housing 102 and the eye. As such, the corneal seal 106 fluidically isolates or substantially fluidically isolates the corneal region 110 from the scleral region 112 in response to the negative pressure, thus minimizing contact of the active agent with the scleral region 112. In other aspects it is also contemplated that the pressure regulator can be at least partially coupled through the housing to both the scleral and corneal regions (not shown), or to the scleral region alone (see FIG. 6). Additionally, the pressure regulator can be located anywhere on the housing with pressure delivering channels connecting to the scleral region, the corneal region, or both the scleral and corneal regions. It is also contemplated that a pressure regulator functionally coupled to only the corneal region can be located in the housing over the scleral region, and alternatively that a pressure regulator functionally coupled to only the scleral region can be located in the housing over the corneal region.

As the negative pressure is applied, the corneal seal provides an initial contact or gripping point that fixes the position of the device as it seals. The location of the strongest contact pressure will thus occur along the corneal seal. This may be contrary to hypothetical devices lacking such a corneal seal that can begin to seal in an arbitrary fashion, and thus may not adhere in the intended position. For hypothetical configurations whereby a vacuum is applied at the center of the cornea, designs lacking a corneal seal may often begin to adhere from the periphery or a portion of the periphery inward in an arbitrary manner. Designs having a corneal seal will grip the eye and adhere around the seal with a vacuum being formed within the sealed region in a predictable manner. As the seal is formed, air pockets are sucked from the corneal region into the pressure regulator as opposed to being forced away from the pressure regulator due to the movement of the housing in response to the negative pressure.

Various designs of corneal seals are contemplated and any such design that is capable of fluidically isolating or substantially fluidically isolating the corneal region from the scleral region is considered to be within the present scope. In one aspect, for example, the corneal seal is an annular seal surrounding or substantially surrounding the cornea. The corneal seal can be a projection or extension of the housing material, or it can be formed separately and attached thereto. When positioning the device in the eye, the corneal seal can initially contact the eye and allow for proper positioning prior to the introduction of the negative pressure between the device and the eye surface. Once the device has been positioned, the negative pressure can be introduced to cause the housing to adhere to the surface of the eyeball, thus fluidically isolating, or substantially isolating the corneal region from the scleral region.

The corneal seal is thus sized and positioned in the housing to encircle or substantially encircle the cornea during use. The corneal seal can be sized and positioned at any location on the eye surface that allows the cornea to be encircled or substantially encircled. In one aspect, the corneal seal is positioned around the cornea's periphery. In another aspect, the corneal seal is positioned to encircle the cornea at a distance of from about 0.01 mm to about 2.0 mm inside the cornea's periphery. In some aspects, the corneal seal can encircle the cornea and a portion of the sclera, however in such cases an amount of active agent may contact this portion of the sclera. Additionally, in one aspect the corneal seal can be a circular or annular ring. In another aspect, the corneal seal can be an elliptical or semielliptical ring. It is contemplated that the corneal seal can be of any shape or configuration that substantially precludes contact between the sclera and the active agent, and any such configuration or shape is considered to be within the present scope.

Numerous configurations of the housings of the devices of the present disclosure are contemplated, for both single-use and multiple-use devices. In one aspect for example, the housing can be configured to allow the eyelids of the subject to close substantially completely thereover. In other words, when the device is in contact with the eye, the subject may be able to blink in a fairly normal fashion. In one aspect, such a device can be configured to resemble a contact lens or a scleral lens. Additionally, for those aspects whereby the negative pressure is introduced into the corneal region, a portion of housing can be disposed over the corneal region to provide the vacuum seal. In one aspect, the housing, or at least the corneal seal, can be configured to conform to the surface of the eyeball. Such a conformation in shape can facilitate the sealing of the corneal seal once the vacuum force is applied.

In another aspect, the housing can be configured to be substantially self-contained. Such a self-contained device would contain all the components used in the delivery of the active agent into the eye. In the case of iontophoretic delivery, for example, the power supply, electrodes, and conductive connections therebetween are contained in the device. In this manner, the device allows simple insertion onto the surface of the eye, and can facilitate substantially normal eye closure and blinking during use. This is particularly advantageous for ocular iontophoresis as it provides an easy-to-use all-in-one device that improves patient compliance, especially, when multiple applications are required. In some aspects, the power supply can be located remotely however.

Various materials are contemplated for use as the housing that can securely contain the various components of the device. In the case of iontophoretic devices, the housing materials or at least a portion of the housing materials associated with the electrodes can have dielectric properties sufficient to maintain these components in electrical isolation. It may be additionally beneficial to utilize materials that provide some level of physical flexibility to avoid damage or irritation to the eye surface. Any material having properties beneficial to the construction of such a device would be considered to be within the scope of the present invention. For example, the housing material may include, without limitation, plastics, metals, composites, Teflon, nylons, polyesters, polyurethanes, polyethylenes, polycarbonates, and the like. Materials such as metals may be utilized that are conductive, and thus would need have dielectric materials incorporated therein in order to maintain electrical isolation between various components of the device if used in an iontophoretic device.

As has been described, the housing can include a pressure regulator to create negative pressure between the device and the surface of the eye. The negative pressure may be strong enough to hold the device in place during blinking. The negative pressure can be introduced in the corneal region, the scleral region, or in both the corneal and scleral regions. As is shown in FIG. 2, for example, the housing 102 can include a pressure regulator 202 associated therewith. In this aspect, the pressure regulator 202 is operable to deliver negative or positive pressure to the corneal space 110. Regardless of location, the pressure regulator can be of various configurations. For example, in one aspect, the pressure regulator can be a port or coupling for the attachment of a pressure generating device such as a syringe (e.g., FIG. 1). In another aspect, as is shown in FIG. 2, the pressure regulator 202 includes an attached pressure bulb 204 (or vacuum bulb) used to generate positive and negative pressure. Thus by squeezing and releasing the bulb, negative pressure is introduced into the space between the eye and the device, such as, for example, into the corneal region. Further squeezing of the bulb can generate positive pressure to allow release of the device from the eye. Thus in this case, the bulb can be made having sufficient internal volume to seal the device with negative pressure and unseal the device following further squeezing. Whether or not a pressure bulb is used, the pressure regulator can be configured for use as a handle to facilitate manipulation of the device before, during, and after positioning on the eye. In some aspects, a flow regulator or valve can be associated with the pressure regulator in order to control the flow of positive or negative pressure. For example, a valve such as a duckbill valve can be disposed between the pressure bulb and the corneal space to maintain the pressure on the corneal space-side of the valve (not shown). In another aspect, the pressure regulator can be positioned in the housing at a location that is off-center relative to the center of the cornea in order to allow the subject to more easily blink or see through the device, as is shown, for example, in FIG. 4. As one specific example, the pressure regulator can be positioned below the midline of the eye in the lower cul-de-sac, a position that allows the upper eyelid to cover a substantial portion of the housing before contacting the pressure regulator. It should be noted that, for FIG. 2 and subsequent figures, reused callout numbers refer to similar or substantially similar components described previously, and in these cases such previous description similarly applies.

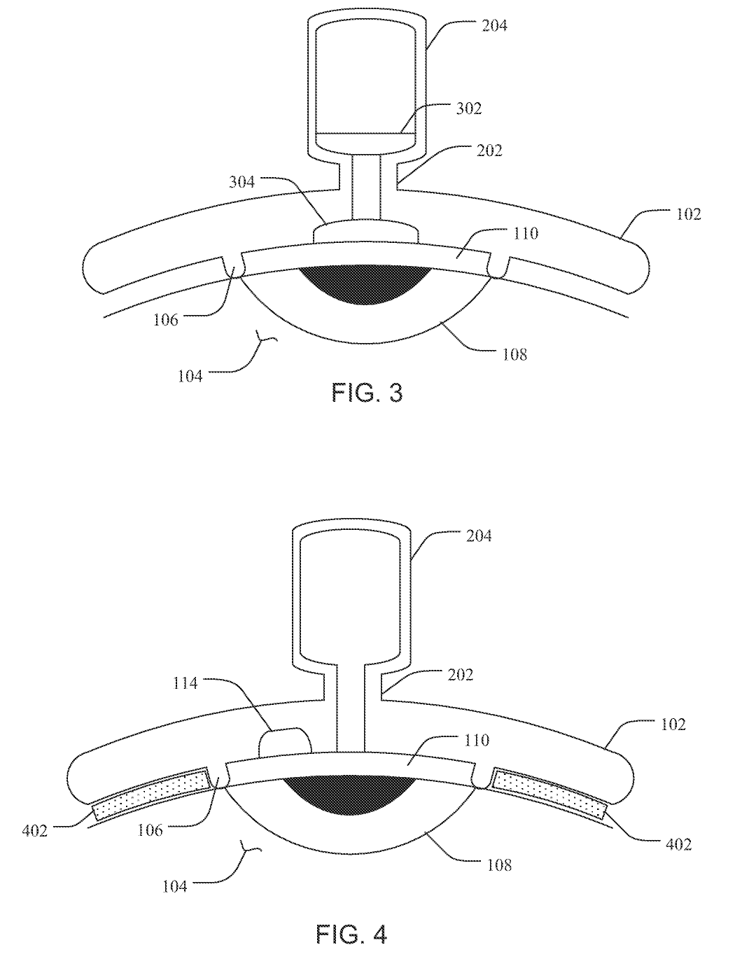

In some aspects, the active agent reservoir can be structurally configured such that the active agent is released into the corneal region or into a secondary reservoir by the activation of the pressure regulator. For example, depressing a pressure bulb can deliver an active agent from the active agent reservoir into the corneal region or into a secondary reservoir near the corneal region. In this way, a device can be preloaded with an active agent and thus be activated as the device is positioned on the eye. In one aspect, as is shown in FIG. 3 for example, an active agent reservoir 302 is associated with the pressure bulb 204 in such a way as to eject active agent therefrom as the pressure bulb 204 is depressed, thus delivering the active agent into the corneal region 110. In some aspects, the active agent can be released into a secondary active agent reservoir 304 near the corneal region 110 to be subsequently delivered therefrom. In some aspects, the secondary active agent reservoir can include a matrix material to retain the active agent in order to moderate delivery. The active agent reservoir can be located within the pressure bulb as shown, or it can be located outside the pressure bulb and associated therewith in such a way that activation causes release of the active agent (not shown). For example, the active agent reservoir can be positioned adjacent to the pressure bulb. The active agent reservoir can also be configured such that releasing the pressure bulb to create the negative pressure within the corneal region functions to draw the active agent from the active agent reservoir. One non-limiting design to achieve this may include separate channels from the pressure bulb and from the active agent reservoir to the corneal space. In other aspects, the active agent can be released from the active agent reservoir by a mechanism that is separate from the pressure bulb. Furthermore, in some aspects the pressure bulb can have a multistage design. For example, a pressure bulb can have at least two distinct pressing regions. One region may be depressed to create the negative pressure between the housing and the eye, while another region can be depressed to release the active agent or to create positive pressure to release the device from the eye.

To further control fluid across the surface of the eye, an absorbent barrier 402 can be positioned under the housing on the sclera surrounding the corneal seal, as is shown in FIG. 4. Various designs are contemplated, and any such designs are considered to be within the present scope. By precluding fluids from the sclera immediately adjacent to the corneal seal, leakage of the active agent into the sclera is further minimized. Additionally, the absorbent barrier will absorb any active agent that does cross the corneal seal and preclude its movement into the sclera.

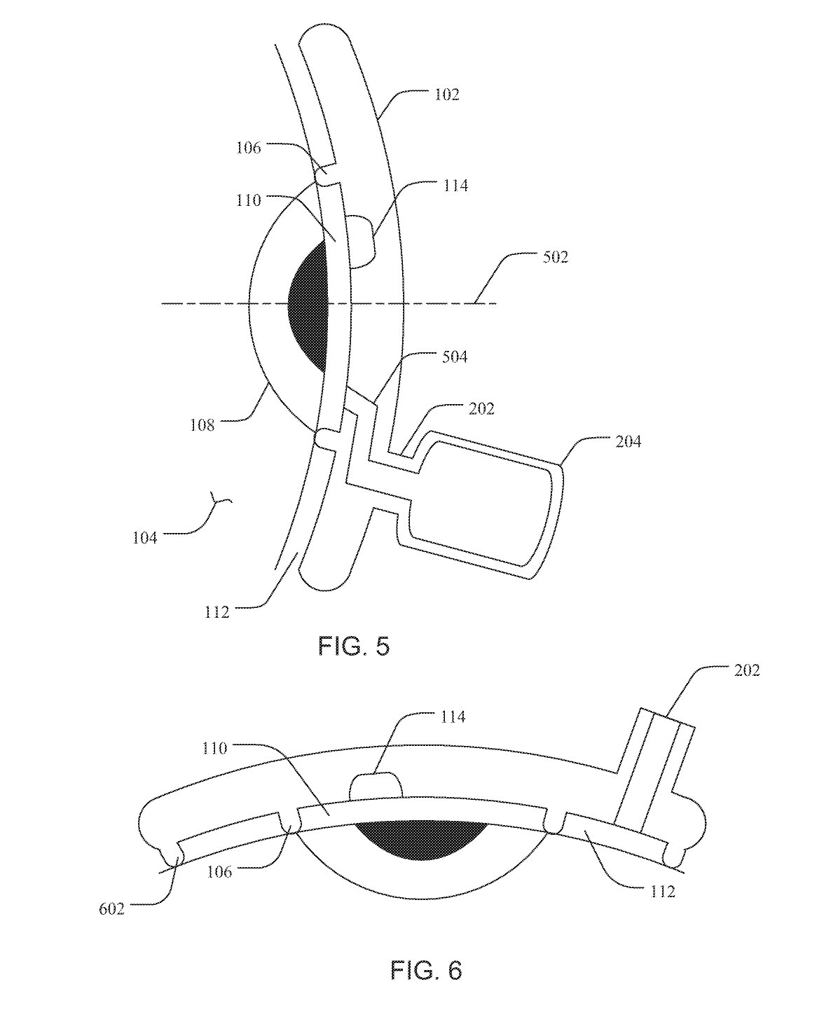

In some aspects, the pressure regulator can be positioned to achieve a variety of desired results related to manufacturing and/or use of the device. As has been described, for example, the pressure regulator, in this case a pressure or vacuum bulb, can be positioned in the housing so as to sit below the horizontal midline 502 of the eye (FIG. 5). When a human subject blinks, the upper eyelid moves to a greater degree than the lower eyelid. Thus, the upper eyelid descends across the eye to meet the lower eyelid. By positioning the pressure bulb 204 below the horizontal midline 502 of the eye, the upper eyelid can more effectively close over the housing 102, thus allowing the subject to blink during delivery of the active agent. While in some cases the pressure bulb 204 may be situated outside of the housing above the corneal region 110 (e.g. over the scleral region), an internal channel 504 can functionally couple the corneal region 110 to the pressure bulb 204 to thus allow a vacuum to be created in the corneal region 110. It should be noted that the positioning of the pressure regulator in this case is merely exemplary, and a variety of other positioning and designs are contemplated, depending on the overall device design and desired delivery results. Additionally, the pressure regulator can be oriented at various angles upon exit of the housing depending on the desired design of the device. In one aspect, the pressure regulator can be oriented at substantially 0 degrees from the midline of the eye. In another aspect, the pressure regulator can be oriented at between about 0 degrees and about 45 degrees from the midline of the eye. In yet another aspect, the pressure regulator can be oriented at between about 0 degrees and about 60 degrees or more from the midline of the eye.

As has been described, in some aspects, as is shown in FIG. 6, the pressure regulator can create the negative pressure between the housing and the eye in the scleral region 112. A scleral seal 602 can be used to facilitate the maintenance of the negative pressure within the scleral region 112. The scleral seal 602 can have the same, similar, or different properties and characteristics as the corneal seal 106.

As has been described, at least one active agent reservoir is associated within the housing in a position to deliver at least one active agent and in some cases at least one secondary compound into the cornea of the eye. The reservoirs according to aspects of the present invention are thus designed to hold an active agent or other secondary compound prior to and during administration into the eye of a subject. In one aspect, a reservoir can be a distinct compartment, having a lumen for holding an active agent or other secondary compound to be delivered. A reservoir can be a recessed portion of the housing, a separate structure coupled to the housing, or any other reservoir configuration capable of containing an active agent or a secondary compound. In some aspects, the reservoir can be located at a single discrete location in the housing. As one non-limiting example, the reservoir can be positioned in the housing to only contact a distinct portion of the cornea of the eye. In other aspects, the reservoir can be located across a broader area of the corneal region. In one non-limiting example, the reservoir can be an annular ring located within the corneal region. In another example, the reservoir can have an arc shape, and thus be associated with an arc-shaped portion of the cornea. Additionally, in one aspect the active agent reservoir surface area can include substantially all of the area of the corneal region. In another aspect, the active agent reservoir has a surface area that is less than or equal to about 75% of the surface area of the corneal region. In yet another aspect, the active agent reservoir has a surface area that is less than or equal to about 50% of the surface area of the corneal region. In a further aspect, the active agent reservoir has a surface area that is less than or equal to about 25% of the surface area of the corneal region. In yet a further aspect, the active agent reservoir has a surface area that is less than or equal to about 10% of the surface area of the corneal region.

Additionally, such a reservoir can contain at least one access port to allow the reservoir to be filled, either before, during, or after contact with the eye surface of the subject. Such a configuration can allow the reservoir to be filled during use as the agent within is depleted. In another aspect, a reservoir can be filled during manufacture of the device with an active agent or other secondary compound to be delivered, particularly in those aspects where the device is intended for a single use. Various reservoir materials are known to those skilled in the art, and all are considered to be within the scope of the present invention. Additionally, the active agent or secondary compound can be included in the reservoir in any form, including, without limitation, a liquid, gelatinous, semi-solid, or solid form. In another aspect the reservoir can consist of a portion of the active electrode, such that an active agent or secondary compound is delivered from the electrode when electrical current is introduced.

The devices of the present invention, and thus the active agent reservoirs themselves, can be configured for active or passive delivery. Passive delivery of an active agent is accomplished by releasing the active agent into the corneal region and allowing passive diffusion to move the active agent into the eye. The active agent can be allowed to freely diffuse throughout the corneal region, or it can be contained in a localized region in proximity to the reservoir. In some aspects the active agent can be formulated for passive delivery using various permeation enhancers and/or passive delivery techniques. Further details regarding passive delivery can be found in U.S. patent application Ser. No. 11/999,266, filed on Dec. 3, 2007, which is incorporated herein by reference.

Active delivery of an active agent can be accomplished by a variety of techniques, including, without limitation, iontophoresis, sonophoresis, and the like. In the case of iontophoresis, for example, the active agent reservoir (and in some cases the secondary agent reservoir) is configured to receive electrical current from an active electrode to thus iontophoretically deliver an active agent or other compound therefrom. A return or inactive electrode is electrically coupled to the subject to complete the electrical circuit. The return electrode can be located either on the surface of the eye or at a location remote from the eye such as the earlobe. In some aspects, the return electrode can be an “active” electrode and be associated with a reservoir to deliver a secondary agent or compound. In such cases, the return electrode and the associated reservoir are likely situated on the surface of the eye. Further details regarding iontophoretic delivery can be found in U.S. patent application Ser. No. 11/414,134, filed on Apr. 27, 2006, which is incorporated herein by reference.

Various placement configurations of electrode/reservoir assemblies are contemplated. For example, in many cases side-by-side electrode/reservoir assembly configurations may be beneficial. Such a configuration may allow effective iontophoresis at a target location while minimizing the extent of the movement of the electrical current in other parts of the body. This is particularly beneficial when administering an active agent to sensitive areas such as the eye, where potential adverse effects may be caused by excessive electrical current passing through particularly sensitive tissues such as the retina in the back of the eye, the optic nerve, etc. Numerous placement configurations are possible, and those discussed herein should not be seen as limiting. In one aspect the electrode/reservoir assembly can be located side-by-side with the return electrode on the cornea. In another aspect, the electrode/reservoir assembly may be located on the cornea and the return electrode can be located on the sclera, in the superior cul-de-sac, or in the inferior cul-de-sac. The preferred site of delivery may depend on the site of drug action in the eye to provide a pharmacological effect.

Prior methods of iontophoretic delivery of an active agent to the eye often locate return electrodes remote from the eye. While such embodiments are considered to be within the scope of the present invention, such configurations are inconvenient and allow various conductive pathways to be formed across the tissues surrounding the eye rather than focused only in the eye per se. Placing both the active and return electrodes in association with the surface of the eye can facilitate the passage of electrical current transsclerally into the eye under the electrodes, particularly when current movement across the surface of the eye is limited. In one aspect, the electrodes can be respectively configured on the surface of the eye such that an electrical circuit is completed substantially within the eye of the subject. In other words, the current between the electrodes passes predominantly through the eyeball tissues rather than into or through the connective tissues surrounding the eye. The active and return electrodes can directly contact the surface of the eye, or they can contact the surface of the eye through an intermediate material or reservoir that is part of the device. In either case, such a “direct” contact between the electrodes and the eye surface may facilitate the focusing of electrical current within the eye.

The relative spacing or the inter-electrode distance between the electrodes can play an important role in determining where an active agent is localized in the eye upon delivery. As such, in accordance with one aspect of the present invention, the electrodes can be spaced at an inter-electrode distance that controls the depth and extent of penetration of the active agent within the eye. Such spacing can focus the electric field more effectively within the eye, thus more effectively delivering the active agent. Increasing the inter-electrode distance will generally cause current to flow deeper into the eye, thus iontophoretically delivering the active agent deeper in some cases. Small inter-electrode distances will cause a more superficial delivery of active agent into the eye. Thus, by altering the physical locations of each of the electrodes relative to one another, and thus the inter-electrode distance between them, the active agent can be delivered to particular regions of the eye at specific depths. As such, the inter-electrode distance may vary depending on the intended delivery location. In one aspect of the present invention, however, the inter-electrode distance may be less than about 40.0 mm. In yet another aspect, the inter-electrode distance may be from about 1 mm to about 10 mm. In a further aspect, the inter-electrode distance may be from about 0.3 mm to about 4 mm.

The active and return electrodes pass current due to a potential difference established there between by a power source. The current acts to move an active agent iontophoretically in a direction that is dependent on the charge characteristics of the active agent and the charge orientation of the potential difference between the electrodes. An active electrode, whether it be an anode or a cathode, is designed to deliver electrical current across an associated reservoir to iontophoretically deliver the active agent located therein. As has been described, in one aspect, one electrode can be an active electrode and the other electrode can be a return electrode for merely completing the electrical circuit. For example, the active electrode can be an anode and the return electrode can be a cathode, or vice versa. In another aspect, both the anode and the cathode can each have an associated reservoir for the delivery of compounds. The compounds can be the same or different, depending on the intended use and/or configuration of the device. In those aspects where the compounds are different, both compounds can be active agents, or one compound can be an active agent and one compound can be a secondary compound or agent that may or may not have a direct therapeutic effect. The anode and the cathode can be of the same or different size relative to each other. Also, the surface area of one or both electrodes can be configured to modify their respective current densities when in use.

The present disclosure also includes methods that involve delivering an active agent into the eye of a subject. In one aspect, for example, a method of delivering an active agent through a cornea of an eye of a subject while minimizing delivery of the active agent through the eye's sclera is provided. Such a method can include applying a corneal seal to the eye to encircle the cornea to form a corneal region, where the corneal seal is coupled to a housing extending outward from the corneal seal over the eye's sclera to form a scleral region. The method can also include applying a negative pressure between the housing and the eye to fluidically isolate the corneal region from the scleral region and delivering an active agent to the corneal region. Thus the corneal seal substantially precludes the active agent from entering the scleral region.

Proper vision care and management can be important to a subjects overall health and wellbeing. In addition, vision care and management can be very important to the success of military operations, including the survival of soldiers in the field, particularly to those operating aircraft or complex weapons. The cornea is a very sensitive tissue of the eye due to its intricate neural network embedded within its surface, and even superficial wounds or minor abrasions to the cornea surface can be constant source of aggravation an individual, including to personnel in a combat situation. Severe corneal pain can hamper the normal functioning of such combat personnel and the recovery time for such personnel to return to duty following eye injuries or relevant ocular surgical procedures. Outside of direct combat duties, military personnel and civilians can suffer eye injuries due to a variety of occupational hazards inherent to their normal course of duties such as the handling of tools and complex machinery.

The anesthetization and management of pain relating to corneal damage and/or irritation can be important to the health and wellbeing of affected individuals. As such, in one aspect a method of anesthetizing a cornea of an eye of a subject while leaving the sclera substantially unanesthetized is provided. Such a method can include applying a corneal seal to the eye to encircle the cornea to form a corneal region, where the corneal seal is coupled to a housing extending outward from the corneal seal over the eye's sclera to form a scleral region. The method can further include applying a negative pressure between the housing and the eye to fluidically isolate the corneal region from the scleral region, and delivering an anesthetic agent to the corneal region to anesthetize the cornea, where the active agent is substantially precluded from the scleral region by the corneal seal. Any known anesthetic that can be administered to the eye is considered to be within the present scope. One exemplary group of active agents can include a vanilloid receptor agonist. In one specific aspect, the vannilloid receptor agonist can be resiniferatoxin (RTX). RTX is a vanilloid receptor agonist that binds to TRPV1 and leads to inactivation of sensory neurons. RTX can cause irritating side effects to tissue surrounding the cornea, and as such, minimization of exposure of the active agent to scleral tissue will decrease such side effects.

In addition to pain management, a wide range of other active agents may be used in the present invention as will be recognized by those of ordinary skill in the art. In fact, any agent that may be beneficial to a subject when administered ocularly may be used. Examples of the active agents that may be used in the treatment of various conditions include, without limitation, analeptic agents, analgesic agents, anesthetic agents, antiasthmatic agents, antiarthritic agents, anticancer agents, anticholinergic agents, anticonvulsant agents, antidepressant agents, antidiabetic agents, antidiarrheal agents, antiemetic agents, antihelminthic agents, antihistamines, antihyperlipidemic agents, antihypertensive agents, anti-infective agents, antiinflammatory agents, antimigraine agents, antineoplastic agents, antiparkinsonism drugs, antipruritic agents, antipsychotic agents, antipyretic agents, antispasmodic agents, antitubercular agents, antiulcer agents, antiviral agents, anxiolytic agents, appetite suppressants, attention deficit disorder and attention deficit hyperactivity disorder drugs, cardiovascular agents including calcium channel blockers, antianginal agents, central nervous system (“CNS”) agents, beta-blockers and antiarrhythmic agents, central nervous system stimulants, diuretics, genetic materials, hormonolytics, hypnotics, hypoglycemic agents, immunosuppressive agents, muscle relaxants, narcotic antagonists, nicotine, nutritional agents, parasympatholytics, peptide drugs, psychostimulants, sedatives, steroids, smoking cessation agents, sympathomimetics, tranquilizers, vasodilators, (3-agonists, and tocolytic agents, and mixtures thereof.

Additionally, further examples of active agents may include steroids, aminosteroids, antibacterials, antivirals, antifungals, antiprotozoals, antimetabolites, VEGF inhibitors, ICAM inhibitors, antibodies, protein kinase C inhibitors, chemotherapeutic agents, immunosuppressive agents, neuroprotective agents, analgesic agents, nucleic acid derivatives, aptamers, proteins, enzymes, peptides, polypeptides and mixtures thereof. Specific examples of useful antiviral active agents include acyclovir or derivatives thereof.

Specific examples of active agents may also include hydromorphone, dexamethasone phosphate, amikacin, oligonucleotides, F_{ab} peptides, PEG-oligonucleotides, salicylate, tropicamide, methotrexate, 5-fluorouracil, squalamine, triamcinolone acetonide, diclofenac, combretastatin A4, mycophenolate mofetil, mycophenolic acid, and mixtures thereof.

Under a number of circumstances, the active agent used may be a prodrug, or in prodrug form. Prodrugs for nearly any desired active agent will be readily recognized by those of ordinary skill in the art. Though any prodrug would be considered to be within the scope of the present invention, examples may include the derivatives of steroids, antibacterials, antivirals, antifungals, antiprotozoals, antimetabolites, VEGF inhibitors, ICAM inhibitors, antibodies, protein kinase C inhibitors, chemotherapeutic agents, immunosuppressive agents, neuroprotective agents, analgesic agents, nucleic acid derivatives, aptamers, proteins, enzymes, peptides, polypeptides, and mixtures thereof. One specific example of a steroid derivative may include triamcinolone acetonide phosphate or other derivatives of triamcinolone acetonide, dexamethasone phosphate. For example, it may be preferable to label a steroid with one or more phosphate, sulfate, or carbonate functional groups, so the prodrug can be effectively delivered into the eye and form a complex with the precipitating ion.

In some cases, ocular treatment may be hampered by the in-vivo movement/clearance of the active agent in the eye. It is therefore contemplated that various means for restricting or slowing such movement may improve the effectiveness of the active agent therapy. In one aspect, the in-vivo movement may be restricted by constriction of the blood vessels exiting an area in which the active agent is being delivered or precipitated. Such constriction may be induced by the administration of a vasoconstricting agent. A vasoconstrictor can be administered actively by iontophoretic or other means, or it can be delivered passively. Specific non-limiting examples of vasoconstricting agents can include a-agonists such as naphazoline, and tetrahydrozoline, sympathomimetics such as phenylethylamine, epinephrine, norepinephrine, dopamine, dobutamine, colterol, ethylnorepinephrine, isoproterenol, isoetharine, metaproterenol, terbutaline, metearaminol, phenylephrine, tyramine, hydroxyamphetamine, ritrodrine, prenalterol, methoxyamine, albuterol, amphetamine, methamphetamine, benzphetamine, ephedrine, phenylpropanolamine, methentermine, phentermine, fenfluramine, propylhexedrine, diethylpropion, phenmetrazine, and phendimetrazine. Vasocontricting agents can be administered either before or concurrently with the administration of the active agent. Though administration of the vasoconstrictor may occur following administration of the active agent, the results may be less effective than prior or concurrent administration. Additionally, in some aspects, the vasoconstricting agent can have the same polarity as the active agent and administered concurrently with the active agent. Similarly, the vasoconstricting agent can have the opposite polarity as active agent, and thus be administered from a return electrode.

It may also be beneficial for the application situs to be sealed with a sealant following delivery of the active agent. This procedure may protect the tissue in which iontophoretic administration occurred. Sealants may include any known to one of ordinary skill in the art, including gels, glues and impermeable polymeric or resinous membranes.

It should be understood that the above-described arrangements are only illustrative of the application of the principles of the present invention. Numerous modifications and alternative arrangements may be devised by those skilled in the art without departing from the spirit and scope of the present invention. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. An ocular device for delivering an active agent through a cornea of an eye of a subject, comprising:
a housing configured to couple to the eye of the subject;
a corneal seal coupled to the housing and positioned in the housing to encircle the cornea during use to form a corneal region, wherein the housing extends outward from the corneal seal to form a scleral region, the scleral region being positioned over the eye's sclera during use;
an active agent reservoir coupled to the housing and positioned to release active agent into the corneal region; and
a pressure regulator coupled to the housing and operable to introduce negative pressure between the housing and the eye, wherein the corneal seal is operable to substantially fluidically isolate the corneal region in response to the negative pressure, thus minimizing contact of the active agent with the eye outside of the corneal region.

2. The device of claim 2, wherein the housing extends outward from the corneal seal to form a scleral region, the scleral region being positioned over the eye's sclera during use.

3. The device of claim 1, wherein the active agent reservoir is fluidically coupled to the pressure regulator, such that the active agent is released into the corneal region as a result of activation of the pressure regulator.

4. The device of claim 3, wherein the active agent reservoir is structurally configured to release the active agent into a secondary active agent reservoir located in the corneal region.

5. The device of claim 3, wherein the pressure regulator is a vacuum bulb.

6. The device of claim 5, wherein the device is configured to release active agent from the active agent reservoir as the vacuum bulb is depressed and the corneal seal is configured to seal to the eye as the vacuum bulb is released.

7. The device of claim 1, wherein the pressure regulator is operable to introduce positive pressure between the housing and the eye to facilitate release of the housing from the eye.

8. The device of claim 1, wherein the pressure regulator is positioned in the housing to introduce the negative pressure into the corneal region.

9. The device of claim 8, wherein the pressure regulator is positioned in the housing sufficiently below a midline of the housing such that the subject can substantially close the eye during deliver of the active agent.

10. The device of claim 1, further comprising an anode and a cathode both positioned to be facing the eye and at least one of the anode and cathode being in fluid communication with the active agent reservoir during use.

11. The device of claim 1, wherein the active agent reservoir contains an active agent.

12. The device of claim 11, wherein the active agent is resiniferatoxin.

13. A method of anesthetizing a cornea of an eye of a subject while leaving the sclera substantially unanesthetized, comprising:
applying a corneal seal to the eye to encircle the cornea to form a corneal region;
applying a negative pressure between the housing and the eye to fluidically isolate the corneal region from the sclera; and
delivering an anesthetic agent to the corneal region to anesthetize the cornea, whereby the active agent is substantially precluded from the sclera by the corneal seal.

14. The method of claim 13, wherein the anesthetic agent is a vanilloid receptor agonist.

15. The method of claim 14, wherein the vannilloid receptor agonist is resiniferatoxin.

16. A method of delivering an active agent through a cornea of an eye of a subject while minimizing delivery of the active agent through the eye's sclera, comprising:
applying a housing having a corneal seal to the eye to encircle the cornea to form a corneal region;
applying a negative pressure between the housing and the eye to fluidically isolate the corneal region from the sclera; and
delivering an active agent to the corneal region, whereby the active agent is substantially precluded from the sclera by the corneal seal.

17. The method of claim 16, wherein applying the negative pressure further includes applying a positive pressure to deliver the active agent into the corneal region followed by applying the negative pressure between the housing and the eye to fluidically isolate the corneal region from the sclera.

18. The method of claim 16, wherein the negative pressure is applied between the housing and the eye in the sclera.

19. The method of claim 16, wherein the negative pressure is applied between the housing and the eye in the corneal region.

20. The method of claim 16, further including applying a positive pressure between the housing and the eye to facilitate release of the housing from the eye.

21. The method of claim 16, wherein delivering the active agent includes delivering the active agent passively to the corneal region.

22. The method of claim 16, wherein delivering the active agent includes delivering the active agent iontophoretically to the corneal region.

23. The method of claim 16, wherein the corneal seal is positioned around the cornea's periphery.
